(19) United States
(12) Patent Application Publication
(10) Pub. No.: US 2022/0409331 A1
(43) Pub. Date: Dec. 29, 2022

(54) IDENTIFICATION OF ELECTROSURGICAL ATTACHMENTS USING COLOR INDICATORS

(71) Applicant: Covidien LP, Mansfield, MA (US)

(72) Inventor: Lewis R. Puterbaugh, Longmont, CO (US)

(21) Appl. No.: 17/749,466

(22) Filed: May 20, 2022

Related U.S. Application Data
(60) Provisional application No. 63/214,385, filed on Jun. 24, 2021.

(51) Int. Cl. A61B 90/92; A61B 18/12; A61B 18/16

(52) U.S. Cl. CPC A61B 90/92; A61B 18/1206; A61B 18/16; A61B 2018/00696

(57) ABSTRACT

An electrosurgical system includes an electrosurgical attachment having a plug with an indicator. The system may also include an electrosurgical generator having a port configured to couple to the plug. The port may include a detection circuit having: a light emitting device configured to illuminate the indicator with a light and an optical module configured to measure the light reflected by the indicator and determine a color of the indicator. The system may also include a controller configured to identify a type of the electrosurgical attachment based on the color of the indicator.

### CROSS-REFERENCE TO RELATED APPLICATION

The present application claims the benefit of and priority to U.S. Provisional Application No. 63/214,385, filed on Jun. 24, 2021. The entire disclosure of the foregoing application is incorporated by reference herein.

### BACKGROUND

### Technical Field

The present disclosure relates to electrosurgical systems. In particular, the present disclosure relates to an electrosurgical generator configured to determine a type of an electrosurgical attachments, i.e., accessory or instrument, having a connector with one or more pins based on a color of one of the pins.

### Background of Related Art

Electrosurgery involves application of high radio frequency electrical current to a surgical site to cut, ablate, desiccate, or coagulate tissue. In monopolar electrosurgery, a source or active electrode delivers radio frequency alternating current from the electrosurgical generator to the targeted tissue. A patient return electrode is placed remotely from the active electrode to conduct the current back to the generator.

In bipolar electrosurgery, return and active electrodes are placed in close proximity to each other such that an electrical circuit is formed between the two electrodes (e.g., in the case of an electrosurgical forceps). In this manner, the applied electrical current is limited to the body tissue positioned between the electrodes. Accordingly, bipolar electrosurgery generally involves the use of instruments where it is desired to achieve a focused delivery of electrosurgical energy between two electrodes.

Existing electrosurgical generators use various methodologies to identify instruments, however, such technologies rely on expensive identifiers, e.g., RFID tags. Thus, there is a need for simplified alternatives for identifying electrosurgical instruments and accessories.

### SUMMARY

According to one embodiment of the present disclosure, an electrosurgical system is disclosed. The electrosurgical system includes an electrosurgical attachment having a plug with an indicator. The system may also include an electrosurgical generator having a port configured to couple to the plug. The port may include a detection circuit having: a light emitting device configured to illuminate the indicator with a light and an optical module configured to measure the light reflected by the indicator and determine a color of the indicator. The system may also include a controller configured to identify a type of the electrosurgical attachment based on the color of the indicator.

Implementations of the above embodiment may include one or more of the following features. According to one aspect of the above embodiment, the electrosurgical attachment may be a return electrode pad. The color of the indicator corresponds to a property of the return electrode pad. The controller is configured to modify operation of the electrosurgical generator based on the property. The property is a size of the return electrode pad. The plug may include a plurality of contacts and the indicator forms at least a portion of one contact of the plurality of contacts. The light emitting device may include a plurality of light emitting diodes. The plurality of light emitting diodes may include a red-light emitting diode, a green-light emitting diode, and a blue-light emitting diode. The optical module may include at least one photodiode. The electrosurgical attachment may be an electrosurgical instrument. The controller may be further configured to modify operation of the electrosurgical generator based on the type of the electrosurgical attachment.

According to another embodiment of the present disclosure, a method for controlling an electrosurgical system is disclosed. The method may include inserting a plug of an electrosurgical attachment into a port of an electrosurgical generator. The method may also include illuminating an indicator of the plug using a light emitting device disposed within the port and measuring the light reflected by the indicator at an optical module disposed within the port. The method may further include determining a color of the indicated based on the light. The method may also include identifying, at a controller, a type of the electrosurgical attachment based on the color of the indicator.

Implementations of the above embodiment may include one or more of the following features. According to one aspect of the above embodiment, the method may also include: configuring, at the controller, operation of the electrosurgical generator based on the type of the electrosurgical attachment. Illuminating may also include cycling through a plurality of light emitting diodes of the light emitting device. Illuminating may further include activating each of a red-light emitting diode, a green-light emitting diode, and a blue-light emitting diode of the plurality of light emitting diodes. The method may also include comparing, at the controller, a color code corresponding to the color of the indicator to a plurality of color codes. The method may further include outputting an error on the electrosurgical generator in response to the color code not matching any color code of the plurality of color codes. The method may additionally include configuring the electrosurgical generator based on a match of the color code to one code of the plurality of color codes. The method may also include retrieving one or more settings associated with the one code of the plurality of color codes. Configuring may include adjusting operation of the electrosurgical generator based on the at least one setting.

### BRIEF DESCRIPTION OF THE DRAWINGS

The present disclosure may be understood by reference to the accompanying drawings, when considered in conjunction with the subsequent, detailed description, in which:

FIG. 1 is a perspective view of an electrosurgical system according to an embodiment of the present disclosure;

FIG. 2 is a front view of an electrosurgical generator of FIG. 1 according to an embodiment of the present disclosure;

FIG. 3 is a schematic diagram of the electrosurgical generator of FIG. 1 according to an embodiment of the present disclosure;

FIG. 4 is a perspective view of a plug of an electrosurgical instrument or accessory and a port of the electrosurgical generator of FIG. 1 according to an embodiment of the present disclosure;

FIG. 5 is a printed circuit diagram of a detection circuit of the electrosurgical generator of FIG. 1 according to an embodiment of the present disclosure;

FIGS. 6A-C is an electrical schematic diagram of the detection circuit of the electrosurgical generator of FIG. 1 according to an embodiment of the present disclosure; and

FIG. 7 is a flow chart of a method for identifying an electrosurgical instrument or accessory coupled to the electrosurgical generator of FIG. 1 according to an embodiment of the present disclosure.

### DETAILED DESCRIPTION

Embodiments of the presently disclosed system are described in detail with reference to the drawings, in which like reference numerals designate identical or corresponding elements in each of the several views. As used herein the term “distal” refers to the portion of the surgical instrument coupled thereto that is closer to the patient, while the term “proximal” refers to the portion that is farther from the patient.

In the following description, well-known functions or constructions are not described in detail to avoid obscuring the present disclosure in unnecessary detail. Those skilled in the art will understand that the present disclosure may be adapted for use with either an endoscopic instrument, a laparoscopic instrument, or an open instrument. It should also be appreciated that different electrical and mechanical connections and other considerations may apply to each particular type of instrument.

An electrosurgical generator according to the present disclosure may be used in monopolar and/or bipolar electrosurgical procedures, including, for example, cutting, coagulation, ablation, and vessel sealing procedures. The generator may include a plurality of outputs for interfacing with various ultrasonic and electrosurgical instruments (e.g., ultrasonic dissectors and hemostats, monopolar instruments, return electrode pads, bipolar electrosurgical forceps, footswitches, etc.). Further, the generator may include electronic circuitry configured to generate radio frequency energy specifically suited for powering ultrasonic instruments and electrosurgical devices operating in various electrosurgical modes (e.g., cut, blend, coagulate, division with hemostasis, fulgurate, spray, etc.) and procedures (e.g., monopolar, bipolar, vessel sealing).

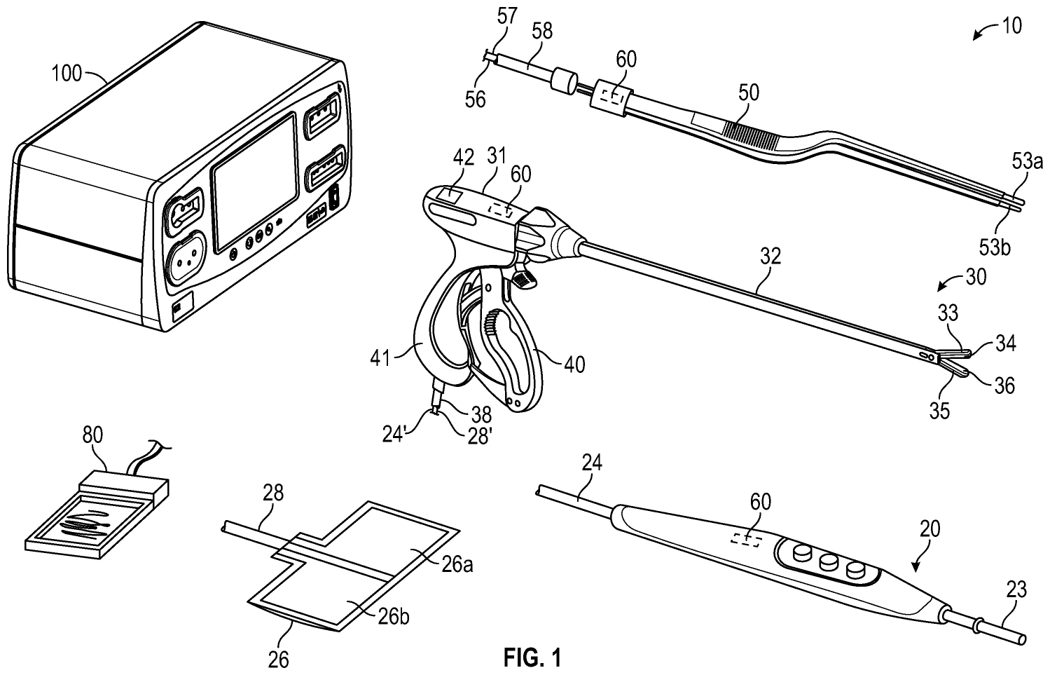

Referring to FIG. 1 an electrosurgical system 10 is shown which includes one or more monopolar electrosurgical instruments, shown as an electrosurgical pencil 20 having an active electrode 23 (e.g., electrosurgical cutting probe, ablation electrode(s), etc.) for treating tissue of a patient. The system 10 may include a plurality of return electrode pads 26 that, in use, are disposed on a patient to minimize the chances of tissue damage by maximizing the overall contact area with the patient. Electrosurgical alternating RF current is supplied to the pencil 20 by a generator 100 via supply line 24. The alternating RF current is returned to the generator 100 through the return electrode pad 26 via a return line 28. In addition, the generator 100 and the return electrode pads 26 may be configured for monitoring tissue-to-patient contact to ensure that sufficient contact exists therebetween. In particular, the return electrode pad 26 includes a pair of foil electrodes 26a and 26b, which are used to monitor tissue-to-patient contact by detecting a difference in electrical properties of the foil electrodes 26a and 26b.

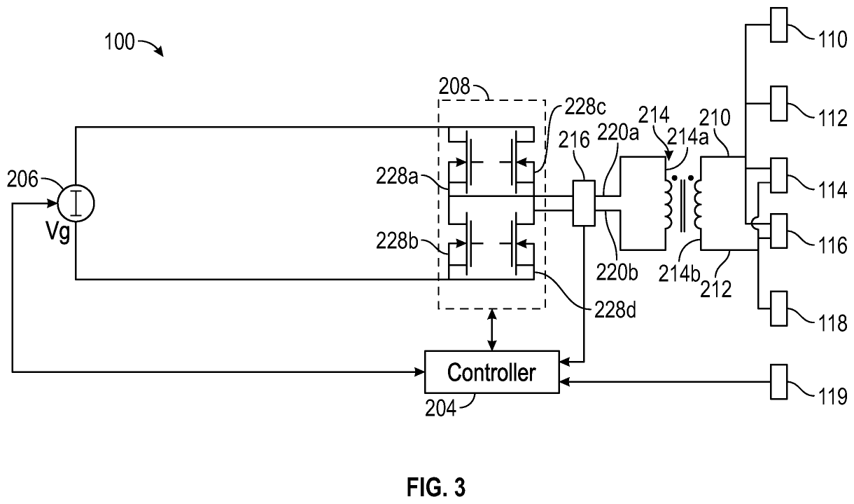

The electrosurgical system 10 also includes one or more bipolar instruments, shown as electrosurgical forceps 30 having one or more electrodes for treating tissue of a patient. The electrosurgical forceps 30 includes a housing 31 and opposing jaw members 33 and 35 disposed at a distal end of a shaft 32. The jaw members 33 and 35 have one or more active electrodes 34 and a return electrode 36 disposed therein, respectively. The active electrode 34 and the return electrode 36 are connected to the generator 100 through cable 38 that includes the supply and return lines 24′, 28′, which may be coupled to the active and return terminals 210 and 212, respectively (FIG. 3). The electrosurgical forceps 30 is coupled to the generator 100 at a port having connections to the active and return terminals 210 and 212 (e.g., pins) via a plug disposed at the end of the cable 38, wherein the plug includes contacts from the supply and return lines 24′, 28′ as described in more detail below. The forceps 30 also includes a button 42 configured to signal to the generator 100 to output electrosurgical energy through the electrodes 34 and 36.

The forceps 30 also includes a lever 40 movable relative to a handle 41. The handle 41 is formed as part of the housing 31 and the lever 40 may be pivotably coupled within the housing 31. The lever 40 actuates, i.e., opens and closes, the jaw members 33 and 35, via one or more mechanical linkages. U.S. Pat. No. 8,784,418, titled “Endoscopic surgical forceps”, provides additional disclosure of a bipolar electrosurgical forceps, the entire disclosure of which is incorporated by reference here. The lever 40 is movable from an open position (i.e., furthest distance from the handle 41) to a closed position (i.e., closest distance from the handle 41). The movement of the jaw members 33 and 35 corresponds to the movement of the lever 40. Thus, the jaw members are movable from an open position (i.e., furthest distance between the jaw members 33 and 35) to a closed position (i.e., closest between the jaw members 33 and 35, clamping tissue).

The electrosurgical system 10 may also include another type of a bipolar electrosurgical instrument, which is shown as tweezers 50 having a pair of electrodes 53a and 53b, respectively, for treating tissue of a patient. The tweezers 50 is coupled to a generator 100 via cable 58 having supply and return lines 56 and 57, respectively.

In addition, the electrosurgical system 10 also include a footswitch 80, which may be a pedal. The footswitch 80 may be paired to activate any one of the pencil 20, the forceps 30, or the tweezers 50 and may provide an alternative activation mechanism in addition to the user inputs on the generator 100 or any hand switches present on instruments.

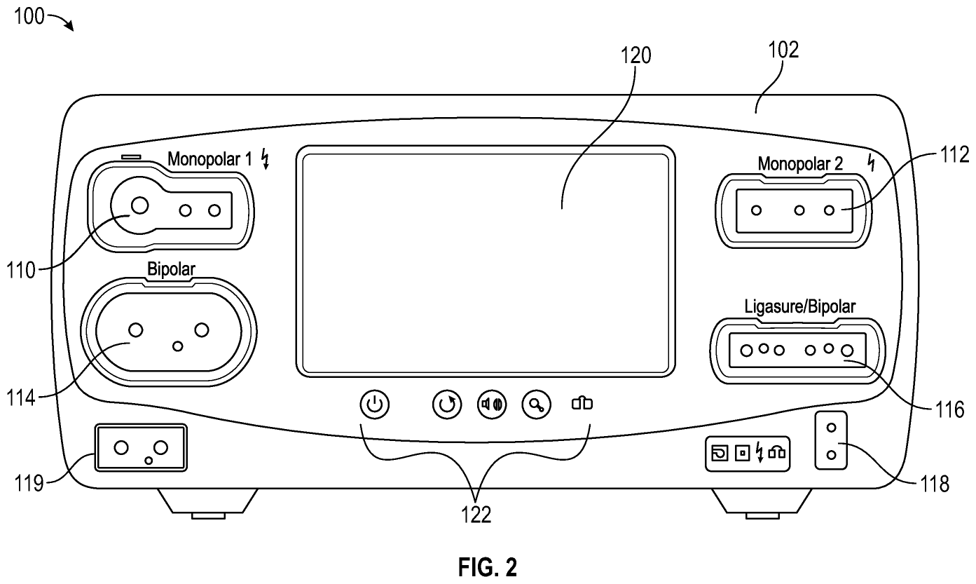

With reference to FIG. 2, a front face 102 of the generator 100 is shown. The generator 100 may include a plurality of ports 110, 112, 114, 116 to accommodate various types of electrosurgical instruments and a port 118 for coupling to a return electrode pad and a port 119 configured to couple to the footswitch 80. The ports 110 and 112 are configured to couple to the monopolar electrosurgical instruments (e.g., electrosurgical pencil 20). The ports 114 and 116 are configured to couple to bipolar electrosurgical instruments (e.g., second electrosurgical instrument 14). The generator 100 includes a display 120 for providing the user with variety of output information (e.g., intensity settings, treatment complete indicators, etc.). The display 120 is a touchscreen configured to display a menu corresponding to each of the ports 110, 112, 114, 116 and the instrument coupled. The user also adjusts inputs by touching corresponding menu options. The generator 100 also includes suitable input controls 122 (e.g., buttons, activators, switches, touch screen, etc.) for controlling the generator 100.

The generator 100 is configured to operate in a variety of modes and is configured to output monopolar and/or bipolar waveforms corresponding to the selected mode. Each of the modes may be activated by the button 42 disposed on the forceps 30. Each of the modes operates based on a preprogrammed power curve that limits how much power is output by the generator 100 at varying impedance ranges of the load (e.g., tissue). Each of the power curves includes power, voltage and current control ranges that are defined by the user-selected intensity setting and the measured minimum impedance of the load.

The generator 100 may operate in the following monopolar modes, which include, but are not limited to, cut, blend, division with hemostasis, fulgurate and spray. The generator 100 may operate in the following bipolar modes, including bipolar cutting, bipolar coagulation, automatic bipolar which operates in response to sensing tissue contact, and various algorithm-controlled vessel sealing modes. The generator 100 may be configured to deliver energy required to power an ultrasonic transducer. Thereby enabling control and modulation of ultrasonic surgical instruments.

Each of the RF waveforms may be either monopolar or bipolar RF waveforms, each of which may be continuous or discontinuous and may have a carrier frequency from about 200 kHz to about 500 kHz. As used herein, continuous waveforms are waveforms that have a 100% duty cycle. In embodiments, continuous waveforms are used to impart a cutting effect on tissue. Conversely, discontinuous waveforms are waveforms that have a non-continuous duty cycle, e.g., below 100%. In embodiments, discontinuous waveforms are used to provide coagulation effects to tissue.

With reference to FIG. 3, the generator 100 includes a controller 204, a power supply 206, and a RF inverter 208. The power supply 206 may be high voltage, DC power supplies connected to a common AC source (e.g., line voltage) and provide high voltage, DC power to their respective RF inverter 208, which then convert DC power into a RF waveform through active terminal 210 and return terminal 212 corresponding to the selected mode. The active terminal 210 and the return terminal 212 are coupled to the RF inverter 208 through an isolation transformer 214. The isolation transformer 214 includes a primary winding 214a coupled to the RF inverter 208 and a secondary winding 214b coupled to the active and return terminals 210 and 212.

Electrosurgical energy for energizing the monopolar electrosurgical pencil 20 is delivered through the ports 110 and 112, each of which is coupled to the active terminal 210. RF energy is returned through the return electrode pad coupled to the port 118, which in turn, is coupled to the return terminal 212. The secondary winding 214b of the isolation transformer 214 is coupled to the active and return terminals 210 and 212. RF energy for energizing a bipolar electrosurgical instrument is delivered through the ports 114 and 116, each of which is coupled to the active terminal 210 and the return terminal 212. The generator 100 may include a plurality of steering relays or other switching devices configured to couple the active terminal 210 and the return terminals 212 to various ports 110, 112, 114, 116, 118 based on the combination of the monopolar and bipolar instruments being used.

The RF inverter 208 is configured to operate in a plurality of modes, during which the generator 100 outputs corresponding waveforms having specific duty cycles, peak voltages, crest factors, etc. It is envisioned that in other embodiments, the generator 100 may be based on other types of suitable power supply topologies. RF inverter 208 may be a resonant RF amplifier or non-resonant RF amplifier, as shown. A non-resonant RF amplifier, as used herein, denotes an amplifier lacking any tuning components, i.e., conductors, capacitors, etc., disposed between the RF inverter and the load, e.g., tissue.

The controller 204 may include a processor (not shown) operably connected to a memory (not shown). The controller 204 is operably connected to the power supply 206 and/or RF inverter 208 allowing the processor to control the output of the RF inverter 208 of the generator 100 according to either open and/or closed control loop schemes. A closed loop control scheme is a feedback control loop, in which a plurality of sensors measures a variety of tissue and energy properties (e.g., tissue impedance, tissue temperature, output power, current and/or voltage, etc.), and provide feedback to the controller 204. The controller 204 then controls the power supply 206 and/or RF inverter 208, which adjust the DC and/or RF waveform, respectively.

The generator 100 according to the present disclosure may also include a plurality of sensors 216, each of which monitors output of the RF inverter 208 of the generator 100. The sensor 216 may be any suitable voltage, current, power, and impedance sensors. The sensors 216 are coupled to leads 220a and 220b of the RF inverter 208. The leads 220a and 220b couple the RF inverter 208 to the primary winding 214a of the transformer 214. Thus, the sensors 216 are configured to sense voltage, current, and other electrical properties of energy supplied to the active terminal 210 and the return terminal 212.

In further embodiments, the sensor 216 may be coupled to the power supply 206 and may be configured to sense properties of DC current supplied to the RF inverter 208. The controller 204 also receives input (e.g., activation) signals from the display 120, the input controls 122 of the generator 100 and/or the electrosurgical pencil 20 and the forceps 30. The controller 204 adjusts power outputted by the generator 100 and/or performs other control functions thereon in response to the input signals.

The RF inverter 208 includes a plurality of switching elements 228a-228d, which are arranged in an H-bridge topology. In embodiments, RF inverter 208 may be configured according to any suitable topology including, but not limited to, half-bridge, full-bridge, push-pull, and the like. Suitable switching elements include voltage-controlled devices such as transistors, field-effect transistors (FETs), combinations thereof, and the like. In embodiments, the FETs may be formed from gallium nitride, aluminum nitride, boron nitride, silicon carbide, or any other suitable wide bandgap materials.

The controller 204 is in communication with the RF inverter 208, and in particular, with the switching elements 228a-228d. Controller 204 is configured to output control signals, which may be pulse-width modulated (“PWM”) signals, to switching elements 228a-228d. In particular, controller 204 is configured to modulate a control signal supplied to switching elements 228a-228d of the RF inverter 208. The control signal provides PWM signals that operate the RF inverter 208 at a selected carrier frequency. Additionally, controller 204 is configured to calculate power characteristics of output of the RF inverter 208 of the generator 100, and control the output of the generator 100 based at least in part on the measured power characteristics including, but not limited to, voltage, current, and power at the output of RF inverter 208.

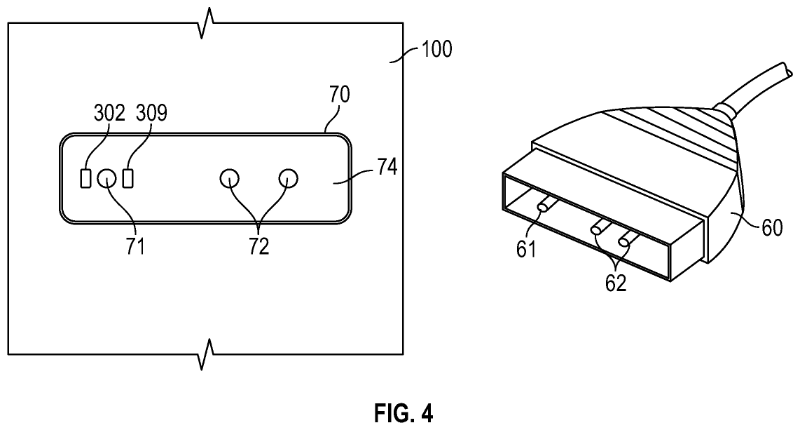

With reference to FIG. 4, each of the pencil 20, forceps 30, tweezers 50, the return electrode pad 2, and the footswitch 80 includes a plug 60 having a plurality of electrical contacts 62, which may be pins, blades, strips, or any other suitable type of electrical contact. The plug 60 is configured to couple to a port 70, which may be one of the ports 110, 112, 114, 116, 118, 119. The port 70 includes electrical contacts 72 configured to couple to the electrical contacts 62 once the plug 60 is inserted into the port 70 to establish an electrical connection.

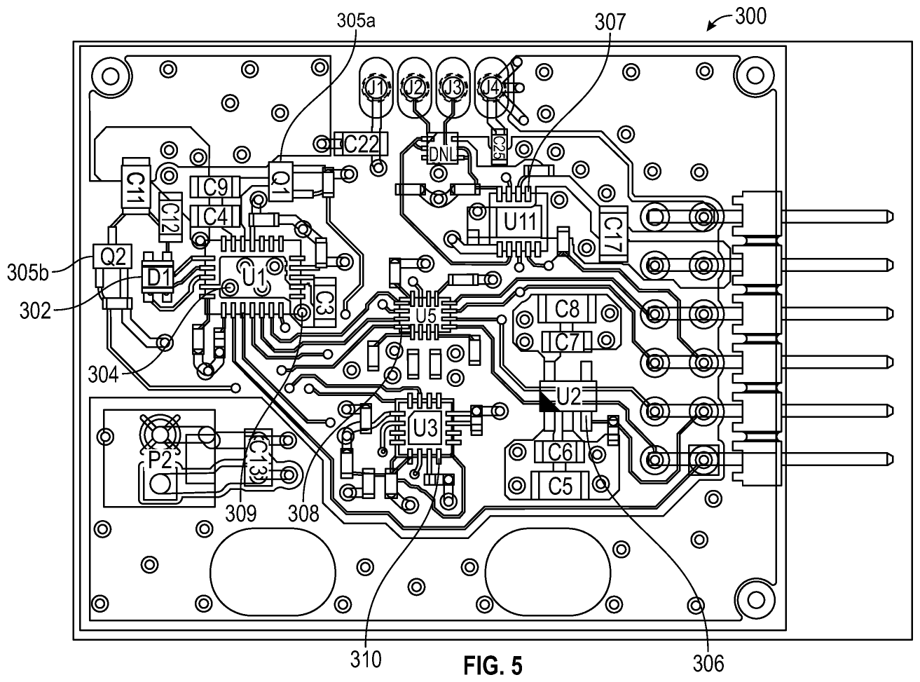

With reference to FIG. 5, the generator 100 includes a detection circuit 300 configured to identify a type of an accessory (e.g., return electrode pad 26, footswitch 80, etc.) or instrument (pencil 20, forceps 30, tweezers 50, etc.) being used. In embodiments, the return electrode pad 26 may have multiple types based on a size of the foil electrodes 26a and 26b, e.g., adult, pediatric, neonatal, since the size of the foil electrodes 26a and 26b depends on the size of the patient. Automatic identification of the size and/or type of the return electrode pad 26 allows the generator 100 to set current limits and other settings based on the type of the return electrode pad 26. Similarly, each of the instruments may include multiple types based on electrode size and/or shape, wire length, which utilize different electrosurgical settings. With respect to the footswitch 80, the generator 100 may configure the number of foot pedals and the functions that are paired to each of the foot pedals. The generator 100 may automatically configure itself based on the type of the instrument or other specifications of the instrument as determined by the detection circuit 300.

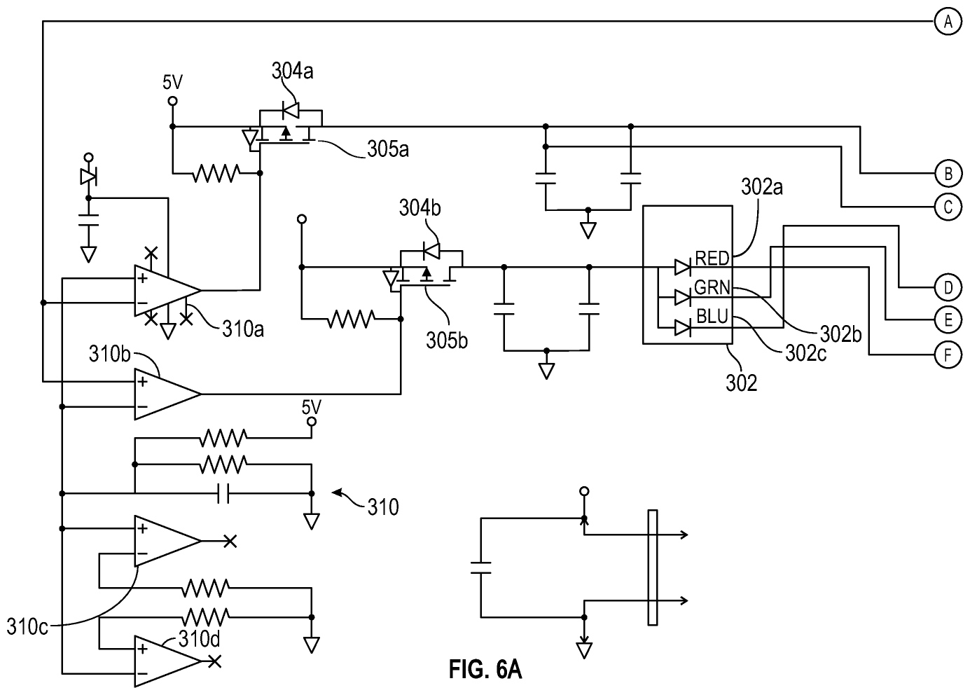

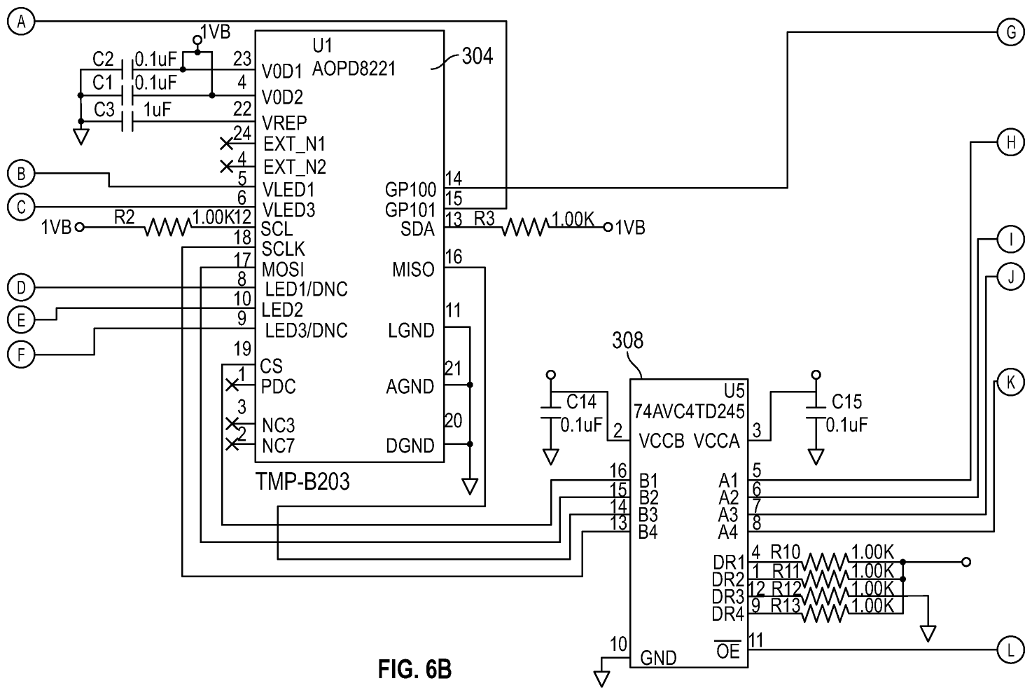

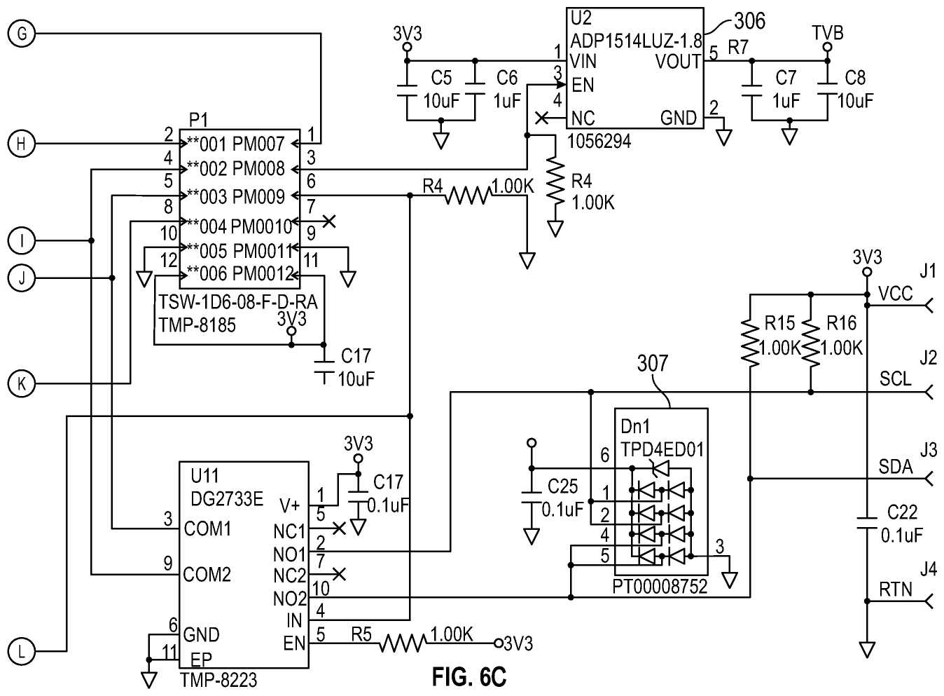

With reference to FIGS. 5 and 6A-C, the detection circuit 300 operates by detecting a color of an indicator 61, which may be a dummy contact (e.g., non-functional) or a functional contact. The port 70 may also include a counterpart contact 71, which may be functional or non-functional. In embodiments, the detection circuit 300 may detect the color of the plug 60 or a portion of the plug 60 surrounding the electrical contacts 62. The detection circuit 300 includes an external light emitting device 302 configured to output visible light having wavelength from about 380 to about 750 nanometers. The light emitting device 302 may include a plurality of light emitting diodes (LEDs) 302a, 302b, 302c. The LED 302a is configured to output red light, the LED 302b is configured to output green light, and the LED 302c is configured to output blue light, such that the combined output is equivalent to white light. In embodiments, the light emitting device 302 may include a single white light LED.

The detection circuit 300 may also include additional internal LEDs 304a and 304b. The LED 304a may be configured to generate blue light and the LED 304b may be configured to generate IR light. The detection circuit 300 may include a pair switching devices 305a and 305b, which may be field effect transistors. The detection circuit 300 also includes a voltage comparator 310 having comparators 310a, 310b, 310c, 310d. Each of the switching devices 305a and 305b is coupled to LEDs 304a and 304b, respectively. The voltage comparator 310 and the switching devices 305a and 305b allow for selection between internal LEDs 304a and 304b and external LEDS 302a, 302b, 302c.

In embodiments, the detection circuit 300 may also include an analog multiplexer 307 or any other suitable switching element configured to couple to other sensor interfaces including, but not limited to, radio frequency (RF) interrogator to read RFID tags or wired interfaces, such as 1-WIRE or I²C interfaces for communicating with EPROM and other storage devices.

The detection circuit 300 includes an optical module 304 configured to drive the light emitting device 302 and make light intensity measurements. The optical module 304 may use a synchronous serial communication interface, such as a serial peripheral interface. A low-drop regulator 306 generate a voltage, which may be about 1.8 V, that a level shifter 308 translates to interface voltage, which may be about 3.3 V. The optical module 304 also include one or more photodiodes 309 configured to measure light. The photodiode 309 may be integrated into the same packaging as the optical module 304, which may be configured as an integrated circuit.

The light emitting device 302 is disposed within the port 70 and is aimed at an indicator 61. The optical module 304 is also disposed within the port 70 such that the photodiode 309 is configured to measure light emitted by the light emitting device 302 that is reflected from the indicator 61. In embodiments, the port 70 includes contact surface 74 on which the contacts 72 are disposed. The light emitting device 302 and the optical module 304 are disposed on the contact surface 74 as well and are positioned such that the indicator 61 at least partially blocks the light emitted from the light emitted device 302 to prevent direct light falling on the optical module 304. In embodiments, the light emitting device 302 and the optical module 304 are aligned where the light emitting device 302 is aimed at or near a base 61a of the indicator pin 61 and the photodiode 309 is in the arc of the light reflecting around the base 61a of the indicator pin 61. The light emitting device 302 is alternated between red, green, and blue, i.e., each of the external LEDS 302a, 302b, 302c is activated sequentially, and the reflected light is picked up by the photodiode 309 while the optical module 304 determines the color of the reflected based on the voltage of the signal from the photodiode 309. The optical module 304 may be calibrated using a grey scale and is configured to identify more than 800 individual colors, allowing for differentiating among that many different types of instruments and/or accessories.

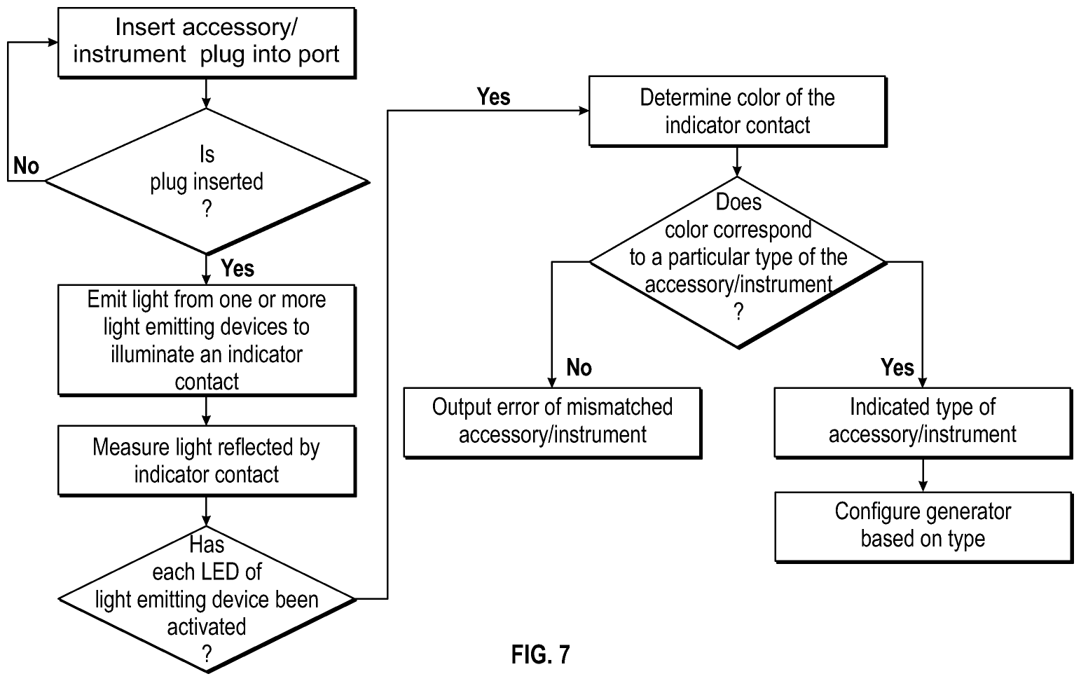

With reference to FIG. 7, a flow chart of a method for identifying an electrosurgical attachment, which may be an accessory (e.g., return electrode pad 26, footswitch 80, etc.) or an instrument (e.g., pencil 20, forceps 30, tweezers 50, etc.). Initially, the plug 60 of the electrosurgical accessory or instrument is inserted into the port 70 of the generator 100. The generator 100 is configured to detect when the plug 60 has been inserted using a limit switch or by supplying a monitoring current through the port 70. Once the generator 100 confirms that the plug 60 has been fully inserted, the controller 204 signals the detection circuit 300 to commence the detection process, which includes cycling each of the LEDs 302a, 302b, 302c of the light emitting device 302 to illuminate the indicator 61. Each activation of the light emitting device 302 includes measuring the reflected light using the optical module 304, which determines the color of the indicator contact 62 based on combination of the measurements for each of the light cycles. Once a color determination is made, a color code is transmitted by the detection circuit 300 to the controller 204 to compare the color code to a list of color codes corresponding to approved list of accessories and/or instruments. If the color code does not match any entries on the list, the controller 204 my output an error on the display 120. If the color code matches, the controller 204 may indicate the type of the accessory and/or instrument as well as automatically configure the generator 100 based on the identified type. In embodiments, automatic configuration may include setting size of the return electrode pad 26 based on the detected size/type of the return electrode pad 26. It is envisioned that the present system may be configured to measure color of multiple color indicators allowing for identification of more attachments.

While several embodiments of the disclosure have been shown in the drawings and/or described herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope of the claims appended hereto.

What is claimed is:

1. An electrosurgical system comprising:
an electrosurgical attachment including a plug having an indicator;
an electrosurgical generator including:
a port configured to couple to the plug, the port including a detection circuit having:
a light emitting device configured to illuminate the indicator with a light; and
an optical module configured to measure the light reflected by the indicator and determine a color of the indicator; and
a controller configured to identify a type of the electrosurgical attachment based on the color of the indicator.

2. The electrosurgical system according to claim 1, wherein the electrosurgical attachment is a return electrode pad.

3. The electrosurgical system according to claim 2, wherein the color of the indicator corresponds to a property of the return electrode pad.

4. The electrosurgical system according to claim 3, wherein the controller is further configured to modify operation of the electrosurgical generator based on the property.

5. The electrosurgical system according to claim 4, wherein the property is a size of the return electrode pad.

6. The electrosurgical system according to claim 1, wherein the plug includes a plurality of contacts and the indicator forms at least a portion of one contact of the plurality of contacts.

7. The electrosurgical system according to claim 1, wherein the light emitting device includes at least one of a plurality of light emitting diodes or a single white light diode.

8. The electrosurgical system according to claim 7, wherein the plurality of light emitting diodes includes a red-light emitting diode, a green-light emitting diode, and a blue-light emitting diode.

9. The electrosurgical system according to claim 1, wherein the optical module includes at least one photodiode.

10. The electrosurgical system according to claim 1, wherein the electrosurgical attachment is an electrosurgical instrument.

11. The electrosurgical system according to claim 1, wherein the controller is further configured to modify operation of the electrosurgical generator based on the type of the electrosurgical attachment.

12. A method for controlling an electrosurgical system, the method comprising:
inserting a plug of an electrosurgical attachment into a port of an electrosurgical generator;
illuminating an indicator of the plug using a light emitting device disposed within the port;
measuring the light reflected by the indicator at an optical module disposed within the port;
determining a color of the indicated based on the light; and
identifying, at a controller, a type of the electrosurgical attachment based on the color of the indicator.

13. The method according to claim 12, further comprising:
configuring, at the controller, operation of the electrosurgical generator based on the type of the electrosurgical attachment.

14. The method according to claim 12, wherein illuminating includes cycling through a plurality of light emitting diodes of the light emitting device.

15. The method according to claim 14, wherein illuminating further includes activating each of a red-light emitting diode, a green-light emitting diode, and a blue-light emitting diode of the plurality of light emitting diodes.

16. The method according to claim 12, further comprising:
comparing, at the controller, a color code corresponding to the color of the indicator to a plurality of color codes.

17. The method according to claim 16, further comprising:
outputting an error on the electrosurgical generator in response to the color code not matching any color code of the plurality of color codes.

18. The method according to claim 17, further comprising:
configuring the electrosurgical generator based on a match of the color code to one code of the plurality of color codes.

19. The method according to claim 18, further comprising:
retrieving at least one setting associated with the one code of the plurality of color codes.

20. The method according to claim 19, wherein configuring including adjusting operation of the electrosurgical generator based on the at least one setting.
